(19) United States
(12) Patent Application Publication
(10) Pub. No.: US 2005/0032118 A1
(43) Pub. Date: Feb. 10, 2005

(54) Nitrophenyl derivatives that can be activated by light

(76) Inventors: Colin H. Self, Ponteland (GB); Stephen Thompson, Newcastle upon Tyne (GB)

Correspondence Address: GREENBERG TRAURIG, LLP, MET LIFE BUILDING, 200 PARK AVENUE, NEW YORK, NY, 10166, US

(21) Appl. No.: 10/812,044

(22) Filed: Mar. 29, 2004

Related U.S. Application Data
(63) Continuation of application No. 08/635,887, filed on Jun. 18, 1996, now abandoned.

(51) Int. Cl. C12Q 1/68; G01N 33/53

(52) U.S. Cl. 435/7.1; 530/350; 435/6; 536/24.3

(57) ABSTRACT

The invention relates to a method of selectively coating a molecule, in a reversible and random fashion, with a plurality of electromagnetically labile compounds so as to modulate the activity and/or availability of said molecule, and to reverse this modulation on exposure to electromagnetic energy. The invention also concerns the provision of a molecule having reversibly and randomly attached thereto a plurality of electromagnetically labile compounds with a view to modulating the activity/availability of said molecule.

The invention relates to the selective modulation of activity and/or availability of molecules and particularly, but not exclusively, of biomolecules especially large biomolecules such as DNA, RNA, receptors, enzymes, substrates, therapeutic agents, hormones, reaction intermediaries, toxins, antigens, conjugates and antibodies.

It is well known by industrial chemists, clinicians and researchers that specific interactions between a number of chemical/biochemical species are determined not only by the inherent interactive nature of said species but also by the ability to bring said species together before other events, reactions or the like, can affect the given species.

In addition, in industrial/research work it is not always possible or practicable to add a predetermined enzyme at a predetermined time to a given chemical reaction so as to ensure that a given reaction sequence is initiated. In these circumstances, it would be highly desirable to be able to add such enzyme at any given time and arrange for its selective activation at a further predetermined time in a given chemical reaction. Further, it would also be desirable for this activation to occur spontaneously in response to the activating signal.

It therefore follows from the above that the ability to switch on or off, or to modulate the activity of, chemical and biochemical molecules would have a major impact on the way industrial, clinical and research reactions were undertaken.

For over a decade, researchers' have investigated the possibility of modulating the activity of molecules. Kaplan et al [Biochemistry 17 1925-1935 (1978) and EP-A-0233403] successfully coupled a photocleavable molecule, that is a single 2-nitrobenzyl residue to a small substrate molecule ie adenosine 5′-triphosphate ATP. As the substrate molecule, ATP, has no inherent activity it would be more appropriate to say that its coupling to 2-nitrobenzyl was a means of making it unavailable as opposed to inactive. On irradiation with UV light the 2-nitrobenzyl residue cleaves from the substrate, thus the substrate becomes available for use in a reaction. Kaplan et al were therefore able to provide a “reservoir” of ATP for use in a given reaction. However, this work is of limited application because it is not possible to use Kaplan's approach for large macromolecules. This is because it is extremely unlikely that a single 2-nitrobenzyl group attached to a macromolecule would affect the activity of the macromolecule. Indeed, the only way this could happen, would be if the 2-nitrobenzyl residue was coupled in a site specific manner to the active site of the macromolecule. This represents an extremely complicated procedure and a prerequisite of the procedure is a precise knowledge of the structure of the active site of the molecule.

Similarly, EP-A-0,233,403 relates to the ‘caging’ of small biochemical molecules having phosphate, thiophosphate, phosphonate, carboxy or phenolic groups, and the patent discloses a family of diazo compounds which may be used for binding to such groups. In most of the examples the molecules concerned are caged inositol triphosphate, and doubly caged fluorescein, are mentioned as by-products in two of the example. In each case, instructions are given on how to convert these by-products to the desired singly caged compounds.

Mendel et al [J. Am. Chem. Soc. 113 2758-2760 (1991)] have investigated modulating the activity of larger molecules such as enzymes and specifically phage-T4 lysozyme (T4L). It is of note, that prior to undertaking their investigations, Mendel et al had a detailed knowledge of the biochemistry of the active site of this enzyme. Indeed, they knew that aspartic acid at position 20 occupied a key position in the binding site of the enzyme and was essential for catalytic activity. These workers were therefore able to use genetic techniques, to introduce an aspartyl Beta-nitrobenzyl ester at position 20 in order to produce a catalytically inactive enzyme. Following irradiation at 315nm the 2-nitrobenzyl group was removed and partial lytic activity was restored (32±3%). These workers were therefore able to show that site specific incorporation of unnatural amino acids into proteins could be used to modulate protein activity. Whilst this work was encouraging it did involve elaborate techniques and, as mentioned, a detailed knowledge of the biochemistry of the active site of the molecule.

Similarly, Goldmacher et al [Bioconjugate Chem. 3 104-107 (1992)] have carried out investigations to modulate the activity of macromolecules, in this case a toxin. The toxin, a ribosome-inactivator known as pokeweed antiviral protein (PAP-S) was conjugated to a targeting molecule such as a monoclonal antibody or a lectin using a single 2-nitrobenzyl derivative. In order to ensure that the conjugate lacked toxicity it was necessary to attach the 2-nitrobenzyl group to the active site of the toxin and therefore, once again, this investigation involved prior knowledge of the active site of the toxin and suitable manipulation to ensure that the 2-nitrobenzyl group was attached to this site.

As will be apparent, detailed knowledge of the active site of a macromolecule can, in the instance of each molecule, involve considerable experimentation. The above techniques are therefore somewhat limited in their application.

In contrast, Willner et al [J. Am. Chem. Soc. 113 3321-3325 (1991)] have used a different approach in order to modulate molecular activity. These workers have randomly coupled azo groups to the enzyme papain. On average, 4.6 molecules of azobenzene-4-arboxylic acid were coupled to each enzyme molecule so producing a conjugate in which the azobenzene moiety was in the cis-configuration. On irradiation with light at 400 nm the azobenzene moiety adopted the corresponding transconfiguration. Since the papain conjugated with the trans-moiety binds substrate better than that conjugated with the cis-moiety a net activation of the enzyme was observed on irradiation. These workers were therefore able to randomly couple azo groups to a molecule in order to regulate its activity. However, it is of note that the azo groups were irreversibly coupled to the enzyme and that both the cis and trans forms of the conjugate exhibited reduced enzyme activity compared to the natural enzyme. Moreover, in the instance where azo groups randomly couple to the active site of the enzyme, activity would be irreversibly destroyed. Thus, using this technique, a fraction of the molecular activity to be modulated would be destroyed and further the overall activity of the molecule to be modulated will be reduced. It can therefore be seen from the above that despite the amount of activity that has been invested in techniques to modulate molecular activity no single reversible technique that can be simply applied to any macromolecule, without having a prior knowledge of the biochemistry of the macromolecule and specifically its active site has been derived. It is therefore an object of the invention to provide a method which is relatively easy to execute and which can be applied to a vast range of macromolecules without the need to firstly determine the biochemistry of the active site of said molecules in order to modulate the activity of same. It is also an object of the invention to provide a product of said method so that the activity of said product can be selectively controlled. It is a more specific object of the invention to provide means for an improved method of treatment both curative and prophylatic of tumours and other disease. The invention stems from the discovery that the activities of a range of biologically important molecules can be altered by the attachment of electromagnetically labile residues in a generally non-site-specific manner, and that the original activities of the molecules can be restored by applying electromagnetic energy in order to remove the residues. Usually, a plurality of labile residues is used and these are generally attached randomly, in the sense that each residue attaches to one of a number of possible binding sites, generally sites which are not active sites of the biologically important molecule. Usually, the effect on the molecule is a diminution in activity, but stabilisation and even activation can also occur, as will be seen below.

In one aspect, the invention provides a molecular composite composed of a core molecule having one or more active sites, and having a plurality of smaller labile residues reversibly attached to the core molecule, the attachment of said labile residues causing an alteration of the ability of the core molecule to provide the activity associated with said active site or sites, the labile residue or residues being dissociable from the core molecule by exposure of the molecular composite to electromagnetic energy so as to result in at least partial restoration of the activity associated with said active site (s).

In this context, the term “active site” should be taken to relate to a site on the core molecule which initiates or participates in, or assists in initiating or participating in, a chemical reaction with which the core molecule is associated.

The invention may be thought of as providing a molecular composite composed of a core molecule having one or more active sites and having one or more labile residues reversibly attached to the core molecule at sites different from and elsewhere than the said active site or sites, the attachment of said labile residue or residues causing an alteration of the ability of the core molecule to provide the activity associated with the active site or sites, the labile residue or residues being able to be dissociated from the core molecule by exposure of the molecular composite to electromagnetic energy so as to result in restoration of the activity associated with the said active site or sites.

Usually, the ratio of the molecular weight of a single labile residue to the core molecule is at least 1 to 10, preferably at least 1 to 30, and more preferably at least 1 to 100. Usually, the molecular weight of each labile residue falls within the range 100 to 400, with that of the core molecule being more than 10,000.

It will be apparent to those skilled in the art that the term, restoration of activity, will have relative meaning depending upon the nature of the molecule, however, in its broadest meaning the term encompasses either the restoration of a molecule to its native state or the restoration of the functional state of the molecule, whether it be total or partial, so that on exposure to conditions where an activity, event or reaction would normally occur the molecule behaves or responds in a functionally predictable manner.

The term “random” is used to indicate that the binding of said compounds to said molecule is a relatively random event in so far as there are a number of sites where, because of the biochemistry, a number of said compounds could bind, and therefore the binding of said compounds to said one or more sites is random.

The reversible and random coupling of said compounds to said molecule will be referred to hereinafter as coating.

In a preferred embodiment of the invention said molecule is ideally a macromolecule and ideally one having biological activity such DNA, RNA, oligonucleotides any proteins such as a receptor, enzyme, hormone, antibody, glycoprotein, lipoprotein, nucleoprotein, phosphoprotein, chromoprotein and indeed any other protein or even a polypeptide. Alternatively said molecule is an enzyme substrate, toxin, antigen, therapeutic agent or conjugate.

The invention has application to any known macromolecule but it is envisaged that it will have specific application to macromolecules possessing at least active properties, albeit directly or indirectly, as opposed to structural or storage properties. For example, it is considered that the invention will be particularly applicable to controlling the activity of macromolecules having molecular weights of 10,000 for example 50,000-400,000 and specifically the activity of macromolecules which interact with other molecules, macromolecules or substrates.

In another aspect, the invention provides a molecular construct for use in the targeted treatment of a localised entity comprising a group of cells within a human or animal body, which entity is distinguished from surrounding tissue by the presence of a specific determinant which is either absent from or present in substantially lower concentration in the surrounding tissue, the molecular construct comprising a targeting portion capable of binding to said specific determinant, and a functional portion capable of causing the release of a biochemically active agent which has an effect on the entity, the molecular construct further comprising at least one labile residue attached by a photocleavable bond either to said targeting portion or to said functional portion, said residues(s) effecting a substantial diminution of the ability of the respective portion to perform its aforementioned function, the residue(s) being dissociable from the remainder of the construct upon exposure to electromagnetic radiation of appropriate energy, to restore at least a substantial portion of said ability.

The molecular construct may for example be used in a modification of the ADEPT methodology which ADEPT methodology is described in EP 0 392 745 and EP 0 540 859. In preferred embodiments the entity to be treated is a tumour, and the functional portion comprises an enzyme capable of converting a substantially non-toxic prodrug into a drug having substantial cytotoxicty: Preferably, the targeting species comprises an antibody, but other site specific agents may also be used, as are set out in more detail below.

The invention also extends to a method of treating a localised entity comprising a group of cells in a human or animal body which entity is distinguished from surrounding tissue by the presence of a specific determinant which is either absent from or present in substantially lower concentration in the surrounding tissue, the method consisting of the following steps, in any order, provided that step a) precedes step c):—
a) administering to said human or animal body a molecular construct comprising a targeting portion capable of binding to said specific determinant, and a functional portion capable of causing the release of a biochemically active agent which has an effect on the entity, the molecular construct further comprising at least one labile residue attached by a photocleavable bond either to said targeting portion or to said functional portion, said residues(s) effecting a substantial diminution of the ability of the respective portion to perform its aforementioned function, the residue(s) being dissociable from the remainder of the construct upon exposure to electromagnetic radiation of appropriate energy, to restore at least a substantial portion of said ability,
b) administering a prodrug capable of being converted by said functional portion to an active drug having a desired therapeutic effect in relation to said entity, and
c) applying electromagnetic energy to the entity to effect dissociation of said residues, said molecular construct and said prodrug being administered in amounts such that said drug is generated in a therapeutically effective amount.

Molecular constructs according to the invention may have the targeting portion deactivated or the functional portion deactivated, or both. An advantage of coating the targeting portion is that this can then be selectively activated in the region of the tissue to be treated, substantially eliminating non-specific binding of the construct at sites in the body away from the location at which electromagnetic radiation is applied. On the other hand, coating the functional portion also has particular advantages. In particular, when a prodrug-activating enzyme is used, deactivation of the enzyme by coating in accordance with the invention allows the level of prodrug in the body to be built up to a higher level than is achievable in the prior art ADEPT method. When a toxin is used as the functional portion of the construct, this must be inactivated.

If one of the moieties is to remain functionally unaffected, and is unaffected by the coating procedure, then coating may be carried out after association without any protection. If, however, coating has an unwanted effect on this moiety or moieties, then coating of the moiety or moieties to be affected should be done (a) before association, (b) if performed after association then the coating conditions may be controlled such that there is sufficient differential inhibition between the moieties, or (c) the moiety or moieties to be unaffected should be protected from unwanted effects of the coating procedure.

If a conjugate between an antibody and enzyme is required in which the enzyme is to remain active but the antibody's activity is to be reversibly inhibited by coating, then it would be possible to: (i) coat the antibody and then conjugate it to the enzyme; (ii) conjugate the antibody and enzyme and then coat the conjugate such that the antibody is inhibited sufficiently for use, but the enzyme remains active enough for use; (iii) coat the conjugate to such extent that both activities are reversibly inhibited; (iv) bind the conjugate to antigen on, for example, an affinity binding reagent such as antigen binding sepharose, carry out the coating procedure and then release the coated conjugate from the affinity binding reagent. There are numerous methods to achieve specific protection—for example: antibody binding to protect an antigen (such as the binding site of a second antibody); protein A to protect an antibody Fc region; substrate (or inhibitor or cofactor) binding to protect the active site of an enzyme.

In some instances, it is desirable for both moieties to be reversibly inactivated. Sequential activation of the moieties may be achieved if different labile residues, for example which dissociate at different frequencies of electromagnetic radiation, are used.

Any cellular marker such as members of the integrin family of cell adhesion molecules, carcino-embryonic antigen, epidermal growth factor receptor, BCA125, CD antigens, T cell receptors which are expressed on either malignant cells, normal cells or any other cell type may be used as targets. Targeting may also be effected against viruses, prions, virus like particle, bacteria and fungi. Ideally the marker will be attached or bound to a functional entity which is coated with electromagnetically labile residues so that the activity of the functional entity is significantly abated until such time as said electromagnetically labile residues are removed. In this way, targeting of the functional entity is achieved in conventional fashion but activation of the functional entity is selectively controlled.

For the avoidance of doubt, cellular markers may, in some instances, be intracellular. For example histochemical application of the invention may favour the use of intracellular markers. Alternatively, intracellular markers may be used in instances involving intracellular research and/or treatments.

As the targeting portion of the molecule any member which binds to the cell can in principle be used. The identity of the targeting portion in any particular case will of course depend on the specific detectable determinants on the cells of the tumour (or other tissue) to be treated, but the following is a non-exhaustive list of molecules which may be used: antibodies and antibody fragments (both natural and synthetic); anti-idiotypic antibodies; other receptor molecules, such as lectins; adhesion molecules, cytokine receptors, hormone receptors and any other such molecules which bind to peptides, polypeptides and other proteins (for example cytokines, such as lymphokines); other small molecules which can bind at specific locations (such as biotin); enzymes (both natural and synthetic, including abzymes); nucleotides, carbohydrates (such as those causing sequestration or other trafficking); and lipids.

Because of the nature of the invention antibodies which would otherwise cross-react with other tissue at other sites can be used. That is to say, because the functional entity of the conjugate is masked or coated the conjugate can in theory bind to any tissue however activation of the conjugate will only occur at the site that is exposed to electromagnetic energy of a wavelength corresponding to an energy capable of cleaving the photocleavable bond between the labile residue and the functional entity.

There are a number of different possibilities for the functional portion of the molecular construct. Of particular utility are enzymes which bind as substrate to a prodrug and convert this to a drug active against the tumour. Typically the prodrug will differ from the active drug by the presence of a protecting group, such as a phosphate group, and the enzyme will therefore typically be a lytic enzyme which removes the protecting group. One example of such an enzyme is alkaline phosphatase, which is capable, for example, of converting inactive etoposide phosphate to active etoposide, inactive mitomycin phosphate to active mitomycin and inactive doxorubicin phosphate to active doxorubicin.

Alternatively, the functional portion may comprise a toxin, such as ricin or diphtheria toxin.

We have also found that the number of labile residues bound to the core molecule can affect the activity of said molecule. For instance, we have discovered that lightly coating a molecule with labile compounds can result in an increase in the activity of said molecule. This is thought to be due to providing an improved environment in and around the active site of said molecule. We have shown this effect with the enzyme chymotyypsin which is a protease. It therefore follows that other proteases may also be affected in this way. A good example of how this knowledge may be used to advantage is in the washing industry where protases are used in washing powders. It follows that a light coating of these proteases may increase the effectiveness of the washing powder. Moreover, it is also postulated that such a light coating may protect the molecule from the denaturing effect of a detergent in a washing powder and/or the temperature of the wash.

It follows from the above that the coupling of labile compounds or residues to said molecule affects the activity of same. Further, the way this activity is affected can be determined in appropriate cases by controlling the number of compounds reversibly bound to said molecule. A small number of labile compounds can confer some activity on the molecule whereas the binding of a large number of said compounds would seem to, to all intents and purposes, inactivate said molecule. The precise number of labile compounds required to bring about the effects may be determined by the size, configuration and/or other properties of the molecule and/or the nature of the compounds themselves. Generally, one or two labile compounds may be required to confer activity on said molecule and between 10 and 15 labile compounds may be required to inactivate said molecule.

Cleavage of said compounds will result either in complete or partial removal of all of said compounds from said molecule or alternatively sufficient removal of a fraction of said compounds in order to restore the molecule to its native state.

Cleavage of said labile compound(s) from the macromolecule (core molecule) is achieved by the use of electromagnetic energy and more specifically the use of UV or visible light.

The invention further provides a method of detecting the presence of a test analyte in a test solution comprising the steps of:
(a) providing a substrate (such as a microtitre plate) having bound thereto a plurality of binding groups, each having at least one accessible active site capable of specifically binding said analyte;
(b) bathing said substrate in a solution containing a plurality of molecular constructs, each comprising a targeting portion capable of binding to said test analyte when said species is bound to a said binding group, and a detectable portion whose presence may be detected either directly or indirectly, at least the targeting portion of each molecular construct having attached thereto one or more labile residue(s), each attached by means of a photocleavable bond, the residue(s) effecting a substantial diminution of the ability of said targeting portion to bind said analyte and being dissociable from the remainder of the construct upon exposure to electromagnetic radiation of appropriate energy;
(c) adding the test solution whose content of test analyte is to be ascertained, so that the test analyte (if present) binds to said binding groups on said sunstrate;
(d) exposing the substrate to electromagnetic energy to cleave the labile residues from said molecular constructs, to enable the constructs to bind to the substrate-bound test analyte, (if present);
(e) flushing unbound molecular constructs from said substrates; and
(f) determining the presence of said detectable portions of said constructs.

Preferably, the binding groups and the targeting portions of the constructs are antibodies raised specifically against the test analyte. The detectable portions of the constructs may either be detectable directly (for example chromophores or radio-labelled species) or indirectly. An example of the latter is an enzyme which converts an added reagent into a form which is readily detectable (for example alkaline phosphatase, which converts p-nitrophenol phosphate to p-nitrophenol which is detectable by standard photometric techniques). The technique may readily be adapted to measure the amount of test analyte by first generating a set of standard results with known concentrations of the test analyte and comparing experimental results with these.

An advantage of this method is that the deactivated molecular constructs can be added before the test analyte, or at the same time, whereas in prior art methods it has been necessary first to bind the test species to the substrate-bound binding groups before adding the targeting/detectable entity. This will lead to a significant simplification of testing methods, and in partcular will make possible simplified test kits which can be supplied complete with the margedigidetecable entity, requiring then only addition of the solution to the teted, irradiation, and detection.

As the electromagnetically labile residue, there can in theory be used any group which can be satisfactorily attached to the core molecule, and which will readily dissociate upon exposure to electromagnetic radiation. Conveniently, the residues are attached to primary or secondary amine groups or carboxyl groups on the core molecule. It is preferred to use residues containing aryl groups, for example those failing within the following general formula: 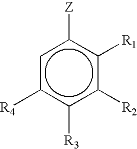
wherein R1=H or NO₂; R₂=H, N₃ NO₂ or OCH₃; R₃=H,OCH₃ NO₂, and R4=H, NO₂ or OCH₃; and preferably wherein at least one of R₁, R₂, R₃ and R₄ is NO₂ and Z is C(R₅)OH— with R₅=H, CH₃, C₂H₅, or an aryl group such as o-nitrobenzyl or phenyl; a glycol such as ethylene glycol an oxycarboxyl group of formula —R₆—O—CO— with R₆=a bond, or a straight or branched lower alkyl group (ie with 1 to 6 carbon atoms, preferably 1 to 3 carbon atoms); an aryl group such as —CO—Y with Y= 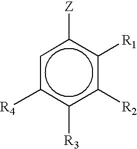
where R=H or CH₃; —S—; or 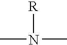
with R,=lower alkyl group, cyclohexyl, or an aryl group such as benzyl or —CH2-C6H6.

Preferred examples of residues may be attached to the core molecule by reaction thereof with a compound selected from the following: 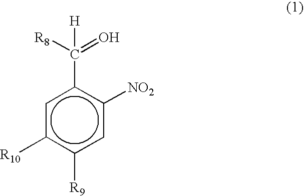
wherein R₈=H, CH₃, C₂H₅; o-nitrobenzyl, or phenyl; and R₉ and R₁₀ are, independently, H or —OCH₃, or sites for irreversible protein or antibody coupling: 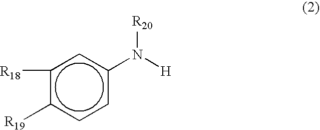
wherein R₁₈ and R₁₉ are, independently, H or —OCH₃, and R₂₀ is CH₃, C₄H₉, cyclohexyl, benzyl or phenly-CH₂—.

A particularly preferred group is a residue derived from the compound labelled (1) above, namely:— 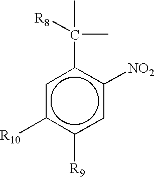
with R₈=H,CH₃ or C₂H₅ and R₉ and R₂₀=OCH₃.

These groups can be produced by swing with the corresponding alcohol, with the hydroxyl group attached to the free carbon atom in the above formula.

The information and features described in a review by V. N. R. Pillai entitled Photoremovable Protecting Groups in Organic Synthesis and published in 1980 by Georg P M Thiene Publishers relating to photoremovable protting groups belong to the description of the invention conained in this application and thus to the content of this application, in so fr as such information and features contribute to achieving the technical aim of the invention, and as such comprise the solution of the technical problem underlying the invention which is the subject of this application. It follows that protection may be sought herein for such features described in this review.

The reaction to attach the labile residues is particularly facilitated by carbonylating the derivative so as to provide an extremely active carbonyl group. The production of such an active derivative enables the binding of said compound to a molecule to take place in relatively simple reaction conditions. More specifically the compound is attached to an amino group on a protein and this results in the random binding of said compounds to said molecule.

This protocol can be carried out using the carbonylating molecule carbonyldiimidazole (CDI) but this may normally require that R1 is hydrogen.

Alternatively, the nitrobenzyl derivative may be treated with phosgene to provide carbonylchloride which can then react with said molecule.

The addition of nitrobenzyl compounds to said molecule makes the molecule more hydrophobic and may protect the molecule if it is to be used in hostile environments. For example, we have shown that aryl sulphatase retains its activity in alkaline buffers when it is coated with the α-methyl substituted derivative of 2-nitrobenzyl alcohol. This may be particularly significant where an enzyme would be fully denatured in an organic environment where such a protective coating, although possibly modulating, ie reducing the activity of the enzyme may be acceptable because this effect is compensated by the prolonged activity of the enzyme.

According to another aspect of the invention there is provided a method for modulating the activity of a molecule comprising reversibly and randomly attaching to said molecule a plurality of electromagnetically labile compounds whereby exposure of said molecule to electromagnetic energy results in a change in said compounds and thus the restoration of activity of said molecule.

For the avoidance of doubt the term. restoration of activity, is to be construed according to the explanation attached to the first aspect of the invention.

The above demonstrates that exposure to electromagnetic radiation results in either the restoration of the molecule to its native state or, the restoration of some of the activity of said molecule. The term, native state, is to be construed as a state that has the activity of the unattached molecule

According to another aspect of the invention there is therefore provided a method of restoring a molecule to its original functional state comprising exposing a molecule treated according to the method of invention with electromagnetic energy.

It is apparent from the above that the molecule, such as an enzyme or any other molecule hereinreferred to, can have its activity modulated by reversibly and randomly coupling thereto a preselected number of electromagnetically labile compounds. Moreover, the linkage to the coated molecule of a target specific agent means that such a molecule can be delivered to a predetermined site and subsequently activated by simply exposing the molecule to electromagnetic radiation. This sort of technique has particular application in research and clinical medicine.

Additionally, molecules reversibly and randomly coated with a preselected number of compounds can be used in chemical reactions so as to modulate the activity of said molecule and, in one instance, to determine when, to all intents and purposes, said molecule is activated within a given reaction or a given chain or reactions.

It will be apparent that molecules of the invention may be provided in assay kits and diagnostic kits. According to a further aspect of the invention there is provided an assay kit or diagnostic kit including a compound of the invention.

Preferably said assay kits or diagnostic kits include means or instructions for effecting or indicating, respectively, how the activity of said molecule can be modulated.

An example of one diagnostic kit of the invention is provided herein under the title “Diagnostic use of coated anti-HCG antibody”. This kit is provided for the purpose of illustration only.

The invention also has application in the provision of therapeutic agents and therefore according to a further aspect of the invention there is provided a therapeutic agent including a molecule whose activity is modulated in accordance with the invention. The invention therefore has application in methods if medical treatment and particularly in the provision of therapeutic agents where a disease or condition is treated by exposure to said molecule and specifically the uncoated version of said molecule.

Embodiments of the invention will now be described by way of example only with particular reference to the following figures and tables wherein:

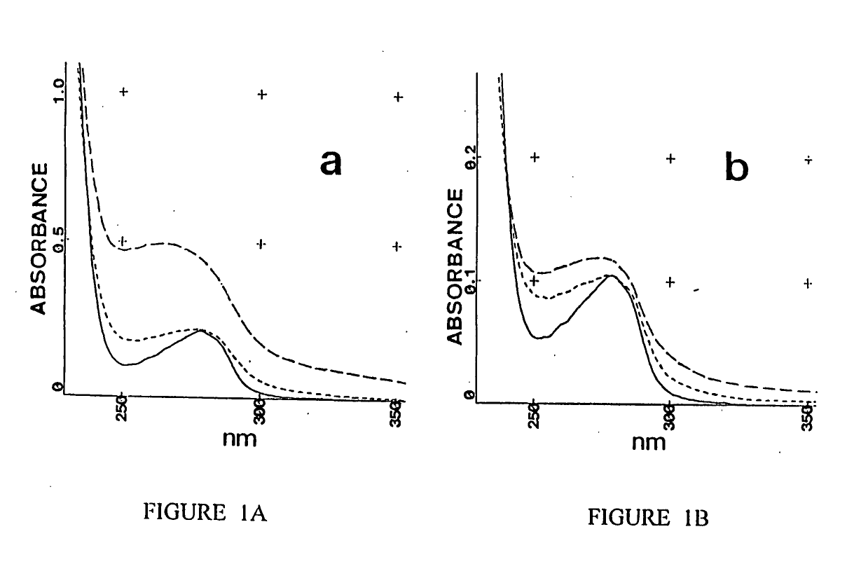

FIGS. 1A and 1B shows the absorbance profile of 1-(2-nitrophenyl)-ethonol-BSA (NPE-BSA) conjugate;

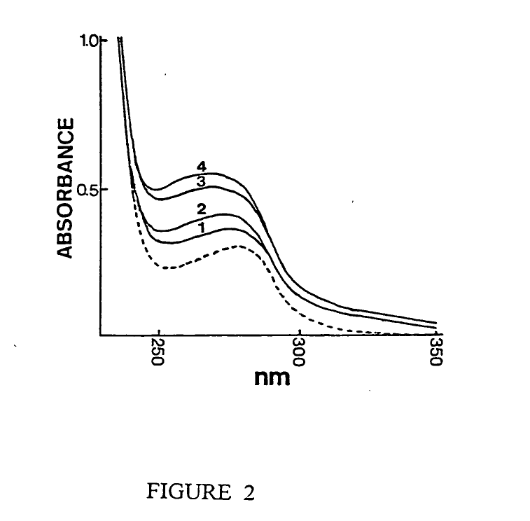

FIG. 2 also shows absorbance profiles of 1-(2-nitrophenyl)-ethonol-BSA (NPE-BSA) conjugates; lines 1, 2 3 and 4 correspond to 3.5, 6.9, 10.8 and 14.5 NPE residues per BSA molecule, respectively, the dotted line shows the absorbance profile of uncoated BSA;

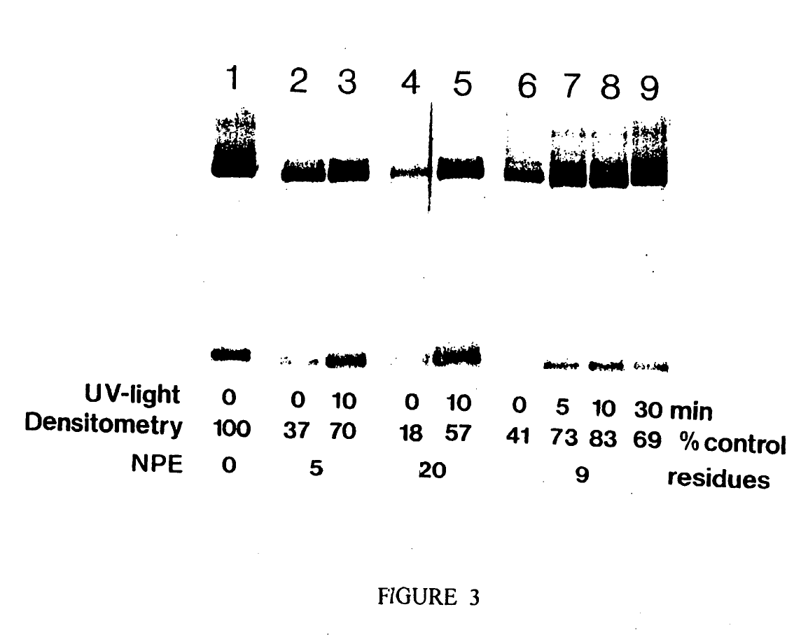

FIG. 3 shows Coomassie blue stained gels that demonstrate the release of human IgG from NPE-IgG complexes on exposure to UV light. Lane 1 shows the amount of IgG bound from a control unlabelled sample, lanes 2 and 3, lanes 4 and 5 and lanes 6-9 show the amount of IgG which bound from three different NPE-IgG preparations before and after treatment with UV light.

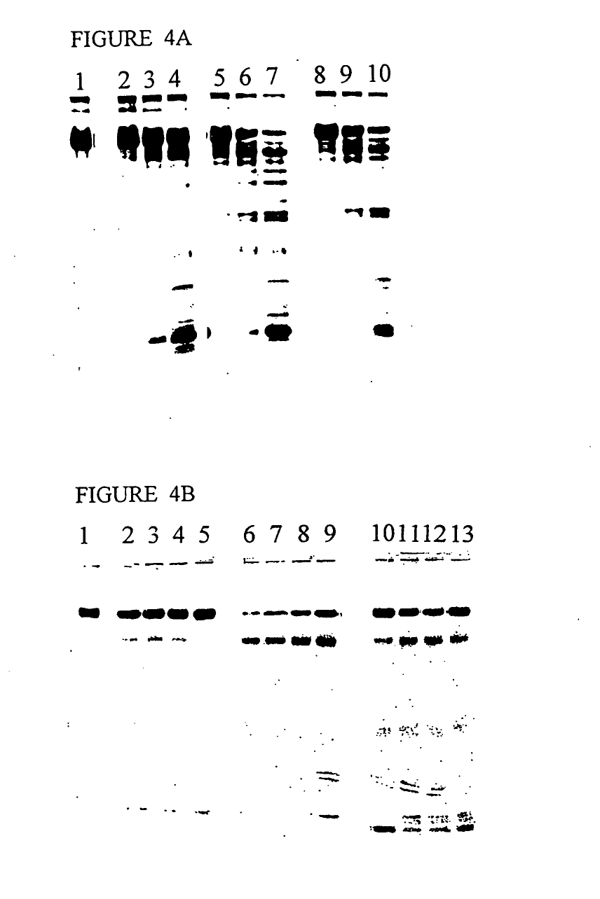

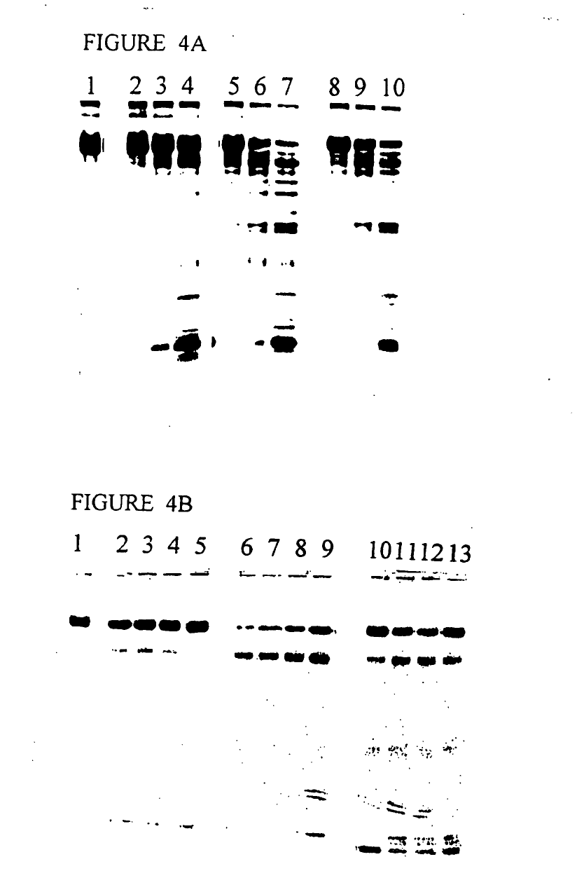

FIGS. 4A and 4B represent polyacrylamide gels showing the activity of NPE-coated and uncoated chymotrypsin:

Table 1 shows the binding of human IgG from NPE-IgG conjugates to anti-IgG antibodies in ELISA assays;

Table 2 shows the binding of goat anti-human IgG to ELISA plates coated with human IgG;

Table 3 shows enzyme activity of aryl sulphatase at a function of coating with NPE groups;

Table 4 shows enzyme activity of aryl sulphatase as a function of coating with NPE groups at pH 6.2 and 7.5.

Experimental Procedures. The coupling of 2-nitrobenzyl alcohol (NBA) and its α-methyl substituted derivative, 1-(2-nitrobenzyl)ethanol (NBE) to proteins.

### Synthesis of 1-(2-nitrobenzyl)ethanol

0.6 g 2-nitroacetophenone (3.6 mmol) was reacted with 0.15 g NaBH4 (3.9 mmol) in 5 ml industrial methylated spirits. TLC in petrol showed that the reaction had gone to completion in 15 mins. Addition of 10 ml acidified water and 10 ml ethyl acetate followed by rotary evaporation of the ethyl acetate layer gave the pure alcohol as a light yellow oil.

### Preparation of 2-nitrobenzyloxycarbonyl-protein conjugates

a) With carboimide. 10 mg (0.065 mmol NBA was reacted with 10 mg (0.062 mmol) of the carbonylating carbodiimide, CDI, in 1.0 ml dry dioxan for 2 hours at 20° C.(1). This solution was then added to 5, 10, or 20 mg bovine serum albumin (BSA) in 4 ml of 0.1M NaHCO₃ pH8.3 and allowed to react for 24 hours at 20° C. This mixtures was dialysed for 16 hours at 4° C. against 0.9% NaCI and then spun at 600 g for 10 min to remove insoluble complexes.

b) With di-phosgene. The procedure of Senter el at (2) was followed. 31.3 μl (0.26 mmol) of DP was added to a solution of 40 mg (0.26 mmol) NBA and 20.6 μl (0.26 mmol) pyridine in 1 ml of dry dioxin. A white precipitate immediately formed and the reaction was shown to go to completion in 15 min by TLC. The reaction mixture was then evaporated in a stream of nitrogen for 45 min to remove unreacted material and the off-white nitrobenzyloxycarbonyl chloride was resuspended in 1 ml of dioxan. Different aliquots of this solution (eg 0. 1, 0.2, and 0.3 ml) were then added to varying concentrations of BSA in 4 ml of 0.1M NaHCO₃ pH8.3 and allowed to react for 24 hours at 20° C. Each mixtures was then dialysed for 24 hours at 4° C. against 0.9% NaCI and white insoluble complexes were removed by centrifugation at 600 g for 10 min.

NBE was coupled to protein by the same procedures as NBA but 11 and 44 mg of NBE were used instead of 10 and 40 mg of NBA.

Photolysis of conjugates. Samples were irradiated in quartz cuvettes at a distance of 0.5 cm with near ultraviolet light for various times at room temperature. After irradiation the samples were dialysed for 16 hours against 0.9% NaCI to remove photocleaved products. A Spectroline EN-16/F ultraviolet lamp (Spectronics Corporation, Westbury, N.Y.) with an emission peak of 365 nm was used as the source of the ultraviolet light. [In early experiments the nitrobenzyl-BSA conjugates were dialysed against phosphate buffered saline both before and after ultraviolet treatment. However, this led to the formation of, what we presume were, colloidal aggregates which could not be centrifuged out of solution even at 13,000 g for 15 min. These particles made the interpretation of spectrophotometry data impossible].

Electromagnetic Sensitivity of Conjugates. Samples of conjugates were irradiated in quartz cuvettes at a distance of 0.5 cm with electromagnetic energy from different regions of the spectrum, specifically from between 312-400 nm. After irradiation the samples were analysed (techniques discussed below) in order to determine the affect of different wavelengths of electromagnetic energy on coating.

We have found that photolabile compounds or residues can be removed from a given molecule using either UV energy or light. This observation is of significance because it means that where a given system, for example a biological system, would be deleteriously affected by UV energy because of it potentially mutagenic effects, natural light could be used to bring about the selective uncoating of a given molecule.

Analysis of conjugates. The absorbence profile of NBA and NBE protein conjugates between 230 and 350 nm were measured on a Unicarn 8700 scanning spectrophotometer. The number of residues of NBA and NBE bound to protein, both before and after exposure to ultraviolet light, was calculated on comparison with the absorbance profiles of BSA, NBA and NBE standards. Protein concentrations were measured using bicinchoninic acid solution (3). The coupling and removal by ultraviolet light of NBA and NBE to proteins was confirmed by isoelectric focusing and electrophoresis in non-denurating conditions.

In the following the invention will be described with reference to NBE.

NBE reacted vigorously with di-phosgene to give a yellowish nitrobenzyloxycarbonyl chloride. This was resuspended in 1 ml of dioxan and 0.25 ml aliquots were added to 25 and 50 mg BSA in 4 ml 0.1 M NaHCO₃. On centrifugation of the dialysed conjugates a large white pellet was obtained but all of the protein remained in solution. Spectrophotometry of diluted portions of the clear supernatants (FIG. 1A and FIG. 1B) showed that there was an average of 14.8 NBE residues bound per protein molecule in the 25 mg sample and 3 NBE residues per protein molecule in the 50 mg sample. On exposure to ultraviolet light for 10 min 14/14.8 (FIG. 1A) and 3/3 (FIG. 1B) of the residues were cleaved from the protein. This proportional substitution of BSA and NBE residues was confirmed in other experiments by reacting 0.1, 0.2, 0.3 and 0.2 ml of the nitrobenzyloxycarbonyl chloride solution with 40, 40, 40 and 20 mg of BSA respectively. The absorbance profiles of the NBE-BSA conjugates (diluted to 0.4 mg/ml) from these experiments are given in FIG. 2. These correspond to 3.5, 6.9, 10.8 and 14.5 NBE residues per BSA molecule. On treatment with ultraviolet light for 10 min. 2.5, 6.0, 10 and 14 of these residues were cleaved. We have also been able to couple up to 11 residues of NBE of ovalbumin by the above technique.

Affect of Coating on Protein Integrity

Experiments to Determine the Reversible Coating of Human IgG with NPE

Nitrobenzlycarbonyl chloride was prepared by treating NPE with di-phosgene in dioxan (2) and aliquots of this (0.5, 20, 100 ul) were added to 1 mg of IgG (Sigma Chemical Co) in 1 ml of 0.1 M NaHCO₃. After dialysis against 0.9% NaCI to remove salts and contrifugation to remove insoluble complexes the NPE-IgG conjugates were obtained as a clear solution. NPE-IgG conjugates (0.2-1.0 mg IgG/ml) were then preabsorbed by gentle overnight rotation with 100 ul Protein A-Sepharose beads to remove IgG molecules which were not coated in the Fc region. The average number of residues of NPE bound to each IgG molecule was estimated by comparing the OD 280 nm values of the NPE-IgG conjugates with NPE and unlabelled IgG standards. Protein concentrations were measured using bicinchoninic acid solution (3). Pre-absorbed NPE-IgG conjugates and unlabelled control IgG were irradiated in quartz cuvettes with UV light from a Spectroline EN-16/F UV lamp with an emission peak of 365 nm at a distance of 0.5 cm. Aliquots (50-100 ul) corresponding to 20-50 ug of IgG were removed at varying times and added to 20 ul of protein A-sepharose beads. After 3 hours incubation unbound NPE-IgG was washed away with 6×2 ml washes of 50 mM Tris-Cl pH 7.4 containing 0.05% Tween20 and bound IgG was eluted by the addition of 100 ul of denaturing electrophoresis sample buffer (4) containing 5% SDS and 8-mercaptoethanol. Equal quantities of sample, 20-50 ul (corresponding to 10 ug unlabelled IgG or 10 ug IgG as NPE-IgG starting material) were then separated by discontinuous electrophoresis in 10% polyacrylamide gels (4). After staining with Coomassie blue the amount of IgG present in each lane of the slab gel was determined by scanning laser densitometry.

NPE was first coupled to varying extents to human IgG (Sigma Chemical Co) as above. The ability of the Fc portion of the IgG to bind to protein A was then studied in the NPE-coated IgG samples. FIG. 3 shows three experiments where pre-absorbed NPE-human IgG conjugates were treated with UV light. There were low initial levels of binding of IgG to protein A (lanes 2,4 and 6) followed by large increases in binding on irradiation with UV light for 5 or 10 min (lanes 3,5,7 and 8). After 30 mins irradiation (lane 9) the binding of IgG dropped, perhaps due to irradiation damage.

These results show that coating of protein, that is antibody, with a photolabile residue is not only reversible but also that the Fc binding region site of the antibody is not deleteriously affected.

This reversible inactivation may be used in both therapeutic and diagnostic applications where it is wished to delay the appearance of antibody Fc effects until a certain time. For example, a diagnostic assay may be conducted in solution (for advantages such as speed of reaction) in a microtitre well coated with protein A, with antigen and Fc-coated antibody. After the antibody antigen reaction has occurred the products of the reaction may then subsequently be bound onto the well by irradiation by UV light, for concentration and measurement.

Use of ELISA Assays to Examine the Availability of Surface Epitopes of Human IgG After NPE Conjugation

100 ul aliquots containing 25. 12.5 and 6.25 ng of IgG (as control IgG or as NPE-IgG conjugates) were added to the wells of an ELISA plate which had previously been coated with a polyclonal goat anti-human Ig antibody (5 ug/ml,overnight). The samples were allowed to react for 2-3 h then unbound IgG/NPE-IgG was washed away. 100 ul of an alkaline phosphatase conjugated goat anti-human IgG(Fc specific) antibody (1/2000 dilution) was added for 1 h, and after further washes p-nitrophenyl phosphate was added as substrate and the absorbance of the yellow product was measured at 405 mn.

Ninety-six well plates were coated with goat anti-human Ig antibody and IgG-NPE conjugates were added, both before and after exposure to UV light for different times. The IgG bound to the first layer antibody was then quantified by the addition of an alkaline phosphatase conjugated polyclonal goat anti-human IgG (Fc specific). The results from a typical plate are shown in Table 1. These results show a decrease in binding in proportion to the number of NPE residues coupled to the IgG and a recovery in binding on exposure to UV light. Values for the recovery in binding capability are even higher when the decrease in the binding of control unlabelled IgG on exposure to UV light is taken into account. On UV irradiation of NPE-IgG conjugates for 10 min there was a drop in binding, possibly due to irradiation damage. Although the table represents the data from only one experiment we have now reproduced similar results on 4 separate occasions.

The results obtained with the ELISA assays complement the results obtained with Protein A columns. The ELISA assays clearly demonstrate that the binding of IgG to polyclonal antisera is markedly reduced when IgG is coated with NPE. The results obtained with the Protein A columns clearly show that the Fc regions of IgG are inaccessible to Protein A when IgG is coated with NPE. On treatment with UV light the NPE residues cleave from the IgG and it then reacts as normal. At certain concentrations IgG binding values from irradiated NPE-IgG conjugates can even exceed those of irradiated controls. Here the NPE coating may protect the underlying IgG from damage during irradiation.

Such UV light-induced changes in the binding of antigen to antibody can have important diagnostic and therapeutic utility. In an anlogous way to the use of Protein A above, the combination of a coated antibody with an antibody against its native form may be delayed until the mixture is irradiated with UV light. Thus, sequence may be attained in diagnostic tests, as with Protein A. In addition, and of particular use in therapeutic applications, activation at a particular location may also be achieved. For example, a coated antibody (which may be linked with cytotoxic agent) may be directed to bind in a specific location by illumination of that location with UV light.

Experiment to Examine the Integrity of IgG ie its Return to its True Native Structure on Removal of NPE

Structural integrity was examined by comparing the Fab-antigen specific binding properties of an antibody on coupling and removal of NPE groups.

Goat anti-human IgG was coated with NPE as described for human IgG. The coated antibody was then pre-absorbed with sepharose beads coated with human IgG to remove antibody that had not been coupled in the Fab region. The NPE-anti human IgG conjugates were then diluted to 0.5 ug/ml and exposed to UV light for 0, 5, 10 and 15 mins. 50, 25 and 12.5 ul of each of these solutions were added to wells of ELISA plate which had previously been coated with human IgG (5 ug/ml, overnight). The samples were allowed to react for 2-3 hr then residual unbound NPE-anti human IgG conjugates were washed away. 100 ul of rabbit anti-goat Ig-alkaline phosphate conjugate (1/2000 dilution) was then added. This reacted with the goat anti-human IgG antibody that had been freed by the UV treatment and had bound to the IgG on the plate. p-Nitrophenol phosphate was added and the absorbance of the yellow product was measured at 405 nm. The absorbance of NPE-anti human IgG conjugates were compared to the absorbance given by the same quantities of unlabelled goat anti-human IgG controls.

An affinity purified goat anti-human IgG (Fc specific) antibody was coated with NPE to an average of 30 NPE residues per Ig molecule. The ability of the conjugate to bind to its specific antigen, both before and after exposure to UV light, was determined using ELISA plates coated with human IgG (Table 2). As little as 0.5-4% of the specific binding of control goat antibody to human IgG remained after the antibody was coated with NPE. The wells which contained the largest amount of NPE-antibody conjugate (50 ul, 25 ng) gave the highest (34%) initial binding values. On irradiation with UV light up to 35% of the antibody's binding capacity returned. The true yield being higher as the binding of control unlabelled goat anti-human IgG samples decreased to around 70% of their original levels upon exposure to UV light (Table 2). The native structure of the Fab binding region of the antibody must, therefore, be conserved during the coupling and subsequent removal of the NPE groups from the antibody. The uncoated goat-anti human IgG antibody was damaged by the UV light to a similar degree as was found for the purified human IgG samples. However, the NPE coated goat anti-human antibody was less damaged by UV light than NPE-IgG conjugates as goat antibody was still being freed and binding to its antigen after 15 mints irradiation (compare tables 1 and 2).

One method of decreasing the varying damages done by the UV light would be to use shorter irradiation times. Considerable amounts of NPE are removed from heavily coated NPE-BSA complexes on 1, 2 and 5 min exposure to UV light (5) and some activity is regained upon irradiation of NPE-IgG for 5 min. Times may further reduce if pulsed light is used. Pulsed laser light is used to released nucleotides from ‘caged’ complexes intracellularly and this doesn't damage the cells under investigation. The amount of damage by the UV light also appears to be protein concentration dependent. In these studies with IgG more damage occurred with lower IgG concentrations. This may be overcome by adding other proteins during irradiation. Goldmacher et al (6) have shown that 7 min exposure to UV light from a lamp source did not damage HeLa cells.

These results show that coating of protein, that is antibody, with photolabile residue does not impair the active site of the protein; integrity is preserved.

### EXAMPLE OF TUMOUR TREATMENT

### Reversibly Inactivated Conjugated Alkaline Phosphatase

Antibody-alkaline phosphatase conjugates are made following the method described in EP-A-0 302 473 A2 Example 1 page 12. These are then coated to inhibit antibody binding activity while retaining enzyme activity, by the method described above. Their reactivity with the prodrug etoposide-phosphate (which may be prepared as described in EP-A 0 302 473 A2 on page 12 line 15 to page 14 line 35) may then be demonstrated. In vitro cytotoxicity with the coated antibody-enzyme complex are investigated as follows: aliquots of H3347 cells (10) are suspended in IMDM growth media (containing 10 ug/ml of the coated conjugate) and all of these aliquots incubated for 30 minutes at room temperature, during the first part of which some should be irradiated with UV light as above. A control cell suspension without added antibody may be also included. The cells should be then washed twice, resuspended in IMDM, and the prodrug added. Incubation at 37° C. is then continued for 15 hours. After washing twice, the cells are plated out and the number of colonies (>8 cells/colony) counted 7-10 days later. Demonstration that significant drug-caused cytotoxicity occurs only in those aliquots which have-been exposed to UV light may be taken further by similar in vivo demonstrations in a manner similar to that described in EP-A-0 302 473 A2 at page 17 lines 21-44, with the coated antibody-enzyme conjugate being used and only one of the bilateral turnouts being irradiated with UV light. The latter shows slower subsequent growth after the prodrug is given than either non-UV irradiated tumours or UV irradiated tumours without either antibody or prodrug.

Dianostic Use of Coated Anti-HCG Antibody

The following is an example of the use of the technique of the present invention in a diagnostic application.

A rabbit polyclonal antibody is raised against human chorionic gonadotrophin (HCG) by standard procedures. The antibody fraction is then purified. The purified antibody is adjusted to a concentration of 50 mg/ml in 50 mM Tris buffer at pH 7.4.

An aliquot of the purified anti-HCG is conjugated to alkaline phosphatase by standard procedure. The conjugate is divided into two aliquots. One is stored and the other is coated with NPE as the example directed to cloaking antibodies described therein.

Wells of a Nunc Microtitre plate are coated with an aliquot of the anti-HCG antibody by diluting it 1:100 in 50 mM bicarbonate buffer pH 9.5 (bb) and putting 200 ul into each well and incubating the plate overnight at room temperature. The solution is then discarded and replaced with a 0.2% solution of bovine serum albumin (Sigma Chamical Co cat. ref. A3803) in BB and then incubated for a further one hour at room temperature. The wells are then washed four times with 200 ul additions of 50 mM Tris pH 7.4 containing 0.02% Tween 20 (TT). 100 ul of a 1:500 dilution of the anti-HCG—alkaline phosphatase conjugate it put into wells of half the micro-titre plate (M). 100 ul of a 1:500 dilution of the NPE coated—alkaline phosphatase conjugate is put into the wells of the remaining hald of the plate (N).

A range of standard HCG (Sigma Chemical Co Ltd cat. ref C2047) samples are then made in T from 40 International Units (IU) in 2-fold serial dilution of 0.02 IU. 100 ul of each standard and a zero standard containing no added HCG is then put into individual wells of the M side of the plate and similarly on the N side of the plate with immediate mixing. The contents of the wells are then uncubated for 40 minutes at room temperature in the dark followed by exposure to electromagnetic radiation, as in the example directed to cloaking antibody described herein, for ten minutes at room temperature and a further ten minutes at room temperature without illumination. The contents of the wells are shaken out and the wells washed four times with TT. The remaining alkaline phosphatase is then determined by addition of the 10 mM p-nitro-phenol phosphate in 50mM bicarbonate buffer pH 10.3 continuing 3.3 mM MgCl₂. The optical density of the wells is regularly monitored at 405 nm and readings recorded when the fastest developing well has reached an optical density of 2.0. Optical density is then plotted against concentration of HCG in the various wells. It is seen that a standard curve which allows the determination of lower levels of HCG is obtained from half N (ie the NPE coated conjugate half) of the plate. Separate samples of human serum containing HCG at low concentration cannot be read by the standard curve of the normal anti-HCG-alkaline phosphatase conjugate whereas it is possible by the standard curve of the NPE-coated anti-HCG-alkaline phosphate conjugate.

Affect of Coating on Enzyme Activity

Experiments to Determine Affect of NPE Coating on Activation of Enzymes

Aryl Sulphatase is an enzyme which removes sulphate groups from sulphated phenol rings. We have coated aryl sulphatase with NPE on 4 separate occasions using our standard reaction buffer (0.1M Bicarbonate pH8.3). An alkaline pH is required to enable the activated NPE groups (NPE-carbonyl chloride) to bind efficiently to the enzyme's lysine residues. The enzyme is unstable at this pH and its reactivity quickly decreases to less than 5% of its original activity. However when NPE-carbonyl chloride is added and the enzyme is coated with NPE, it retains up to around 40% of its original activity despite being coated in a very unfavourable pH environment. Typical values for coating (average number of NPE residues per aryl sulphate molecule) and enzymic activities retained (compared to fresh enzyme made up under optimum conditions) are given in Table 3. The enzyme activity is measured by adding uncoupled and NPE-coated aryl sulphatase (at the same concentration of enzyme) to a solution of 0.01M nitrocatechol sulphate in acetate buffer at pH5.0. After 1-2 hours incubation 1M NaOH is added and the released nitrocatechol becomes dark reddy-brown in colour. The amount of nitrocatechol and hence the enzyme activity can then be determined by measuring the absorption of the solution at 515 nm.

As can be seen from Table 3 some of the enzyme's activity is protected by the NPE coating. (The enzyme is effectively more stable over a larger pH range). If the coating exceeds 10-11 residues the enzymic activity drops either because the enzyme active site is hidden or because the enzyme starts to be denatured.

Experiments of Determine Affect of pH on Coating of Aryl Sulphatase with NPE

We have recently tried to coat aryl sulphatase with NPE at lower pH values (6.2 and 7.5). This causes less damage to the uncoated enzyme but the number of residues of NPE that can be coupled to the enzyme drops. 39% of the enzyme's original activity remains in buffer at pH 6.2, this increases to 49% when it is coated with NPE. 23% of the enzymes activity remain at pH 7.5, this increases to 55% when it is coated with NPE.

These results show that coating of a protein, that is an enzyme, with photolabile residues affords protection/stabilisation to the enzyme and so enables it to function in hazardous environments.

Experiments to Determine Affect of NPE Coating on Activation of Chymotrypsin

Chymotrypsin (2 mg in 1 ml) was reacted with 0, 20 and 50 ul of NPE-carbonyl chloride. 0, 3.6 and 4.4 NPE residues coupled per chymotrypsin molecule on the first occasion and 0, 2.9 and 5.6 residues bound on the second occasion. The proteolytic activity of the NPE-coated chymotrypsin was measured (and compared to uncoupled chymotrypsin) by examining its ability to digest BSA in polyacrylamide gels. BSA was chosen as it is a well characterised and inexpensive protein, but any protein could be used.

10 or 20 ul (5 or 10 ug) of BSA was added to every well of a polyacrylamide gel and varying amounts of chymotrypsin or NPE coated chymotrypsin were added to selected wells. The gels were then electrophoresed at 6 mA fr 1 hour (this low current keeps the BSA and chymotrypsin in contact and allows digestion to proceed), then at 40 mA for 3 hours to separate the digested BSA components. The results from two experiments showing the digestion of BSA with varying amounts of chymotrypsin/NPE-chymotrypsin (gel A) and the effect of UV light on the amount of digestion (gel B) are given in FIGS. 4a and 4b.

In FIG. 4A all lanes contain 20 ul (10 ug) of BSA. Lane 1 is a control lane containing BSA alone. Lanes 24 contain 1, 5 and 25 ug of uncoated chymotrypsin, lanes 5-7 contain 1, 5 and 25 ug NPE (2.9 residues)-chymotrypsin, and lanes 8-10 contain 1.5 and 25 ug NPE (5.6 residues-chymotrypsin, respectively.

Analysis of this polyacrylamide gel shows that the coated enzyme has much more enzyme activity at all three concentrations.

It is also clear that more BSA is digested by the NPE-chymotrypsin coated with 2.6 residues than by the NPE-chymotrypsin coated with 5.6 residues although both these preparations are at least twice as active as uncoated chymotrypsin.

In FIG. 4B, all lanes contained 10 ul (5 ug) of BSA. Lane 1 is a control lane containing BSA alone. Lanes 2-5 contain 20 ul (10 ug) of uncoated chymotrypsin, lanes 69 contain 10 ug NPE (2.9 residues)-chymotrypsin and lanes 10-13 contain 10 ug NPE (5.6 residues)-chymotrypsin. Each sample (lanes 2-5, 69 and 10-13) was irradiated for 7, 15 and 30 mins with UV light respectively.

This gel again shows that the NPE coated enzyme has much more enzyme activity.

Less BSA is digested as unlabelled chymotrypsin is irradiated by UV light (lanes 2-5). Much more BSA is digested by NPE-chymotrypsin (2.9 residues, lane 6) but this markedly reduces towards uncoated values as the NPE is removed by exposure to UV light (lanes 6-9). The NPE chymotrypsin conjugate coated with 5.6 residues gets more active (lanes 10-11) then less active (lanes 11-13) as the UV light takes the more heavily coated chymotrypsin through its more active lightly coated forms to its less active uncoupled structures.

There is therefore a level of coating which markedly increases the activity of chymotyypsin.

It is envisaged that this level will vary according to the nature of the protein to be coated and the residues with which it is coated. But selective and controlled coating can clearly be used to advantage to optimise the activity of a given enzyme.

The activated chymotrypsin may be employed in therapeutic applications such as in preparations provided for individuals with cystic fibrosis.

### SUMMARY

We have shown that it is possible to randomly and reversibly bind a considerable number of nitrobenzyl residues to proteins. With BSA as a model protein if less than 8 residues are bound, then all the residues cleave on exposure to light. If 8-15 residues are bound then about 95% of NBE residues, or 65% of NBA residues are removed on irradiation with light for approximately 10 mins.

The di-phosgene coupling procedure worked well and is probably applicable for the coupling of all small molecules with hydroxyl groups to proteins. However, the carbodimide, CDI only couples the primary alcohol NBA to protein. No coupling of the secondary alcohol, NBE, to protein could be detected.

Our work also shows that the ability of NPE to modulate, or inhibit, the activity of a molecule particularly a protein is completely reversible without altering the structural integrity of the protein. Specifically, experiments using antibodies showed that antibody activity could be reversibly blocked using NPE without compromising the integrity of the Fc or Fab (antigenic) binding sites of the antibodies. These results are of considerable significance when using the invention to regulate cellular and biochemical processes. Moreover, our results also show that the selective coating of a molecule with a predetermined number of residues of NPE can be used to advantage to modulate, typically enhance, the activity of an enzyme and also to protect an enzyme in potentially hostile environments. Our results thus have application in controlling enzyme activity and also prolonging the life expectancy of a given enzyme in a given environment.

Additional Potential Uses of the Invention

It will be apparent to those skilled in the art that the invention has application in modulating the availability or activity of many sorts of protein for example antibodies or even antigens. The invention lends itself to the improved targeting of antibody mediated therapies such as cancer therapies and the directed use of immunotoxins and targeted-enzymes followed by a pro-drug.

For example, if an immunotoxin were to be coated with NPE then the specificity of the immunotoxin should increase as the antibody portion would only bind where the coating was removed that is where UV or light was shone. Non-specific cross-reactions with normal tissue should be eliminated. The need therefore the for antibody to be highly specific for the target cell is eliminated. In addition, or alternatively, the potency of the toxin could be reduced by coating with NPE until irradiated at the target site. In this way, higher doses of immunotoxin could be used in order to treat a specific condition.

Similarly, the coating of an antibody-enzyme conjugate with NPE would also have considerable advantages. As above, there would be more specific antibody binding and less cross reactivity. In addition, excess antibody-enzyme would not have to be cleared from the body prior to the addition of a pro-drug and a higher target dosage of therapeutic agent should result. Moreover, coating with NPE may also have an additional advantage in that it may reduce a patients immune response to any non-human enzyme that may be used.

Another important area where the invention has application is in the regulation of DNA transcription. NPE-blocking residues could be used to regulate the activity of DNA plasmid vectors, especially where UV-insensitive bacterial host strains are used. The photoregulation of DNA function could have immense potential if where required exposure to light, that is UV light and the potential for corresponding damage could be avoided. For example the use of NPE-coated vectors may allow cells to recover from the effects of transfection and multiply before a transfected vector is switched on. This application has tremendous potential in the field of recombinant technology.

Our invention, involving the reversible and random coupling of an electromagnetically labile compound to a given molecule is not intended to be limited by the above examples but is rather intended to be exemplified by them.

References

1) Bethell, G. S., Ayers, J. S., Hancock, W. S., and Hearn, M. T. W. (1979) A novel method of activation of cross-linked agarose with 1,1′-carbonyldiimidazole which gives a matrix for affinity chromatography devoid of additional charged groups. J. Biol. Chem 254,2572-2574.
2) Senter, P. D., Tansey, M. J., Lambert, J. M., and Blattler, W. A. (1985) Novel photocleavable protein crosslinking reagents and their use in the preparation of antibody-toxin conjugates. Photochem. Photobiol. 42, 231-237.
3) Smith, P. K., Krohn, R. I., Hermanson, G. T., Mallia, A. K., Gartner, F. H., Provenzano, M. D., Fujimoto, E. K., Goeke, N. M., Olsen, B. J., and Klenk, D. S. (1985) Measurement of protein using bicinchononic acid. Anal. Biochem. 150, 7685.
4) Thompson S and Maddy A H. In Red Cell Membrances—A Methodological Approach, Young J D and Ellory J C eds pp67-93, Academic Press, New York. (1982).
5) Thompson S, Spoors J A, Fawcett M-C, Self C H. Biochem Biophys. Res. Comm. 201.1213-1219, (1994).
6) Goldmacher V S, Senter P D, Lambert J M, Blattler W A, Bioconjugate Chem. 3, 104-107 (1992).

### EXAMPLES OF NPE-COATED CONJUGATES AND HOW THEY COULD BE UTILISED ADVANTAGEOUSLY

1. Antibody-Alkaline Phosphatase Conjugates in Which the Antibody is Coated With NPE.

The amine groups of an antibody could be coated with NPE then the coated antibody linked via its sulphydryl groups to alkaline phosphatase (using a heterobifunctional linker such as SMCC, Mahan et al Anal. Biochem 162,163-170). Alternatively the Fc portion of the antibody could be protected with protein A, the complex coated with NPE, then disassociated with a low pH. The NPE coated antibody could be separated from the protein A by gel filtration due to its larger size. Only the-Fab-antigen binding region of the antibody would then be blocked with NPE and its uncoated Fc region could be bound to alkaline phosphate using any amine or sulphydryl coupling reagent.

Therapeutic Use

A simple tissue culture model system could be used to show how an NPE-coated antibody in an antibody-alkaline phosphate conjugate could be used to target specific cells.

The first stage would be to grow monolayers of a human colon carcinoma cell line (such as LS 174T) in plastic culture dishes. An antibody which binds to the tumour cell line such as an anti-CEA monoclonal would also be required (Meyer et al. Cancer Res. 53,3956-3963). Alkaline phosphatase would then have to be conjugated to the anti-CEA antibody. If the uncoated anti-CEA-Alkaline phosphatase conjugate were to be added to the cells in the flask, excess conjugate were to be washed away, then a pro-drug(mytomycin phosphate, Etoposide phosphate) added in fresh media, then all of the cells would die. The alkaline phosphatase bound to the cancer cells by the anti-CEA antibody converting the harmless phosphorylated pro-drug to a potently active drug-(mytomycin, Etoposide). This alkaline phosphate mediated DEPT (antibody directed enzyme pro-drug targeting) system how been shown to work by Senter et al (PNAS,85,4842-4846, Cancer Res. 49,5789-5792).

If the same procedure were to be carried out using anti-CEA-Alkaline phosphate conjugate in which the anti-CEA had previously been coated with NPE then no cells would die. The conjugate would be incapable of binding to the cells and would be washed away. If, however, certain areas of the cell monolayer were irradiated with UV light for a few minutes in the presence of the coated conjugate, then the NPE groups would cleave from the antibody, and the conjugate would localise/adhere to the cells in the irradiated areas. After washing, and addition of pro-drug in fresh media, only the cells which had been irradiated would die. In this way specific areas of cells would be killed. As added advantage is that the antibody would not have to be so highly specific for the cancer cells as is currently required, as the main targeting would be light directed.

2. Coat the Enzyme in an Antibody-Chymotrypsin Conjugate

Bagshawe et al have synthesised several pro-drugs which as glutamic acid esters are much less toxic than the native drugs, used in conjunction with the enzyme carboxypeptidase G2 which cleaves the glutamic acid-drug bond, these pro-drugs can be used in ADEPT therapy for cancer. If these or similar pro-drugs were synthesised as tyrosine or tyyptophan esters, then the enzyme chymotrypsin could potentially be used as the activating enzyme in ADEPT therapy as it cleaves aromatic esters.

a. Therapeutic Use

The enzyme fraction of an antibody-enzyme conjugate could potentially be reversibly inhibited in the same way as mentioned above for the antibody portion. We have already shown that the enzyme chymotrypsin can be reversibly inhibited by an NPE coating. LS 174T cancer cells could be grown in monolayers and the anti-CEA-chymotrypsin conjugate added. After washing and addition of the appropriate pro-drug all the cells would be killed. However if the enzyme was first inactivated with an NPE coating, then despite the fact that the conjugate would still bind to all the cells, no cells would be killed when the prodrug was added. The anti-CEA-chymotrypsin conjugate would only be capable of killing the cancer cells of the conjugate coating the cells were to be activated by irradiation by UV light.

The reversible inhibition of the enzyme portion of an antibody-enzyme conjugate may be even more important than the reversible inhibition of the antibody. One degree of specificity would arise through the initial antibody binding followed by another degree of specificity in the killing of the targeted cells through activation of the enzyme and conversion of the pro-drug

b. Diagnostic Use

If an ELISA plate were coated with an antibody to retinal binding protein (RBP) then a second antibody to RBP conjugated to chymorrypsin were added then no results would be obtained as the chymotrypsin (being a protease) would destroy not only the proteins in the sample, but also the capture RBP antibody bound to the plate. However if a second antibody conjugate was added in which the chymotrypsin was unactivated with an NPE coating then a sandwich would be formed with the RBP caught between the capture antibody and the second antibody-chymotrypsin conjugate. After washing and irradiation with UV light a chymotrypsin sensitive chromaphore could be added and the amount of chymotrypsin and hence the amount of RBP quantitated.

1. A molecular composite composed of a core molecule having one or more active sites, and having a plurality of smaller labile residues reversibly attached to the core molecule, the attachment of said labile residues causing an alteration of the ability of the core molecule to provide the activity associated with said active site or sites, the labile residue or residues being dissociable from the core molecule by exposure of the molecular composite to electromagnetic energy so as to result in at least restoration of the activity associated with said active site (s).

2. (Canceled)

3. (Canceled)

4. (Canceled)

5. (Canceled)

6. (Canceled)

7. (Canceled)

8. (Canceled)

9. (Canceled)

10. (Canceled)

11. (Canceled)

12. (Canceled)

13. (Canceled)

14. (Canceled)

15. A product or a method according to claim 1 wherein the electromagnetically labile residue comprises: 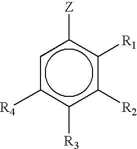
wherein R₁=H or NO₂; R₂=H, N₃ NO₂ or OCH₃; R₃=H, OCH₃ NO₂ and R₄=H, NO₂ or OCH₃; and preferably wherein at least one of R₁, R₂, R₃ and R4 is NO₂; and Z is C(R₅) OH— with R₅=H, CH₃, C₂H₅, or an aryl group such as o-nitrobenzyl or phenyl; a glycol such as ethylene glycol an oxycarboxyl group formula —R₆—O—CO— with R₆=a bond, or a straight or branched lower alkyl group (ie with 1 to 6 carbon atoms, preferably 1 to 3 carbon atoms); an aryl group such as —CO—Y with Y= 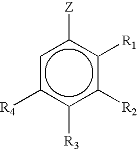
where R=H or CH₃; —S—; or 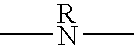
with R,=lower alkyl group, cyclohexyl, or an aryl groups such as benzyl or —CH2-C6H6.

16. A product or a method according to claim 1 wherein said electromagnetically labile residue comprises 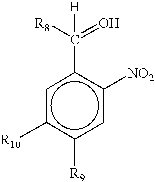
wherein R₈=H, CH₃, C₂H₅; o-nitrobenzyl, phenyl; and R₉ and Rio are, independently, H or —OCH₃, or sites for irreversible protein or antibody coupling.

17. A product or a method according to claim 1 wherein said electromagnetically labile residue comprises 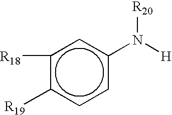
wherein R₁₈ and R₁₉ are, independently, H or —OCH₃, and R₂₀ is CH₃, C₄H₉, cyclohexyl, benzyl or phenly-CH₂—.

18. A product or a method according to claim 1 wherein said electromagnetically labile residue comprises 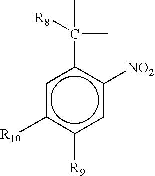
With R₈=H, CH₃ or C₂H₅ and R₉ and R₂₀=OCH₃.

19. An antibody to which is attached a labile residue or residues which reduce the ability of the antibody to bind to its binding partner, the labile residue or residues being able to be disassociated from the antibody by exposure to electromagnetic radiation to restore binding ability of the antibody.

20. An antibody according to claim 1, wherein the labile residue is 2-nitrobenzyloxycarbonyl.

21. The antibody according to claim 19 wherein the labile residue or residues is 2-nitrobenzyloxycarbonyl.

22. The composite according to claim 1 wherein the electromagnetic radiation is visible or uv light.

23. The antibody according to claim 19 wherein the electromagnetic radiation is uv light.
